(19) United States
(12) Patent Application Publication
(10) Pub. No.: US 2016/0214953 A1
(43) Pub. Date: Jul. 28, 2016

(54) PROCESS FOR THE PREPARATION OF DAPAGLIFLOZIN

(71) Applicant: SUN PHARMACEUTICAL INDUSTRIES LIMITED, Mumbai, Maharashtra (IN)

(72) Inventors: Suresh Babu JAYACHANDRA, Hyderabad (IN); Devendra Prakash NAGDA, Udaipur (IN); Tarun Kumar SINGH, Lucknow (IN)

(21) Appl. No.: 15/024,087

(22) PCT Filed: Sep. 18, 2014
(86) PCT No.: PCT/IB2014/064639

§ 371 Date: Mar. 23, 2016

(30) Foreign Application Priority Data
Sep. 23, 2013 (IN) 2801/DEL/2013

(51) Int. Cl. C07D 309/10

(52) U.S. Cl. CPC C07D 309/10

(57) ABSTRACT

The present invention provides an improved process for the preparation of dapagliflozin.of Formula (II) wherein the process comprises the step of hydrolyzing the compound of Formula (III) in the presence of an amine base.

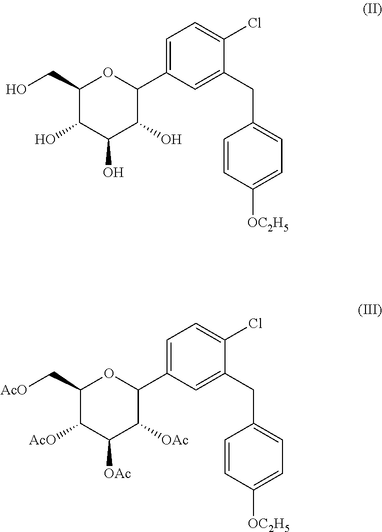 [SMILES: CCOc1ccc(Cc2cc(C3O[C@H](CO)[C@@H](O)[C@H](O)[C@H]3O)ccc2Cl)cc1 ; CCOc1ccc(Cc2cc(C3O[C@H](COC(C)=O)[C@@H](OC(C)=O)[C@H](OC(C)=O)[C@H]3OC(C)=O)ccc2Cl)cc1]

### FIELD OF THE INVENTION

The present invention provides an improved process for the preparation of dapagliflozin.

### BACKGROUND OF THE INVENTION

Dapagliflozin propanediol monohydrate is chemically designated as (1S)-1,5-anhydro-1-C-[4-chloro-3-[(4-ethoxyphenyl)methyl]phenyl]-D-glucitol, (S)-propylene glycol, monohydrate and is marketed for the treatment of type 2 Diabetes mellitus. Its chemical structure is represented by the following Formula I.

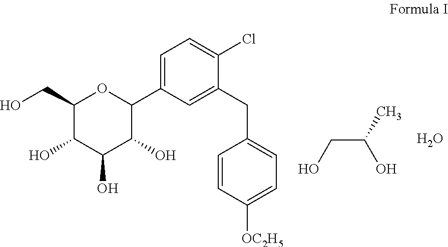

U.S. Pat. Nos. 6,515,117, 7,375,213, 7,932,379, and 7,919,598 disclose processes for the preparation of dapagliflozin comprising the step of hydrolyzing an acetylated dapagliflozin, represented by Formula III, in the presence of an alkali metal hydroxide such as lithium hydroxide or sodium hydroxide. Dapagliflozin obtained from these processes has a significant level of an impurity detected at a relative retention time (RRT) of 1.61 when measured by high performance liquid chromatography (HPLC).

The present invention provides an improved process to minimize or remove this process-related impurity during the manufacture of dapagliflozin.

### SUMMARY OF THE INVENTION

A first aspect of the present invention provides an improved process for the preparation of dapagliflozin of Formula II,

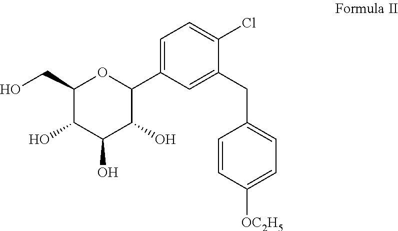 [SMILES: CCOc1ccc(Cc2cc(C3O[C@H](CO)[C@@H](O)[C@H](O)[C@H]3O)ccc2Cl)cc1]

wherein the process comprises the step of hydrolyzing the compound of Formula III

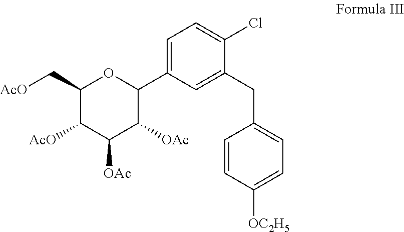 [SMILES: CCOc1ccc(Cc2cc(C3O[C@H](COC(C)=O)[C@@H](OC(C)=O)[C@H](OC(C)=O)[C@H]3OC(C)=O)ccc2Cl)cc1]

in the presence of an amine base.

A second aspect of the present invention provides dapagliflozin substantially free of an impurity detected at a RRT of 1.61 when measured by HPLC.

### BRIEF DESCRIPTION OF THE FIGURES

FIG. 1 depicts the X-Ray Powder Diffraction (XRPD) pattern of dapagliflozin produced by the process of the present invention.

FIG. 2 depicts the Differential Scanning calorimetry (DSC) pattern of dapagliflozin produced by the process of the present invention.

### DETAILED DESCRIPTION OF THE INVENTION

The term “about”, as used herein, refers to any value which lies within the range defined by a number up to ±10% of the value.

The term “substantially free of the impurity detected at a RRT of 1.61”, as used herein, refers to dapagliflozin or its solvates having less than about 0.8%, preferably less than about 0.5%, and most preferably, less than about 0.1% of the impurity detected at a RRT of 1.61, when measured by HPLC. The term “substantially free of the impurity detected at a RRT of 1.61” also includes dapagliflozin or its solvates having no detectable amount of the impurity.

In the context of the present invention, “solvates” refers to complexes of dapagliflozin with water, methanol, ethanol, n-propanol, propanediol, and butynediol.

The compound of Formula III is hydrolyzed in the presence of an amine base. Examples of amine bases include ammonia, methylamine, dimethylamine, triethylamine, tert-butyldimethylamine, phenylethylamine, and diisopropylamine.

In an embodiment of the present invention, the hydrolysis can be carried out in the presence or absence of a solvent. Examples of solvents include water, alcohols, chlorinated hydrocarbons, aromatic hydrocarbons, nitriles, and mixtures thereof.

In another embodiment of the present invention, the hydrolysis of the compound of Formula III is carried out in the presence of methylamine and methanol to obtain the compound of Formula II.

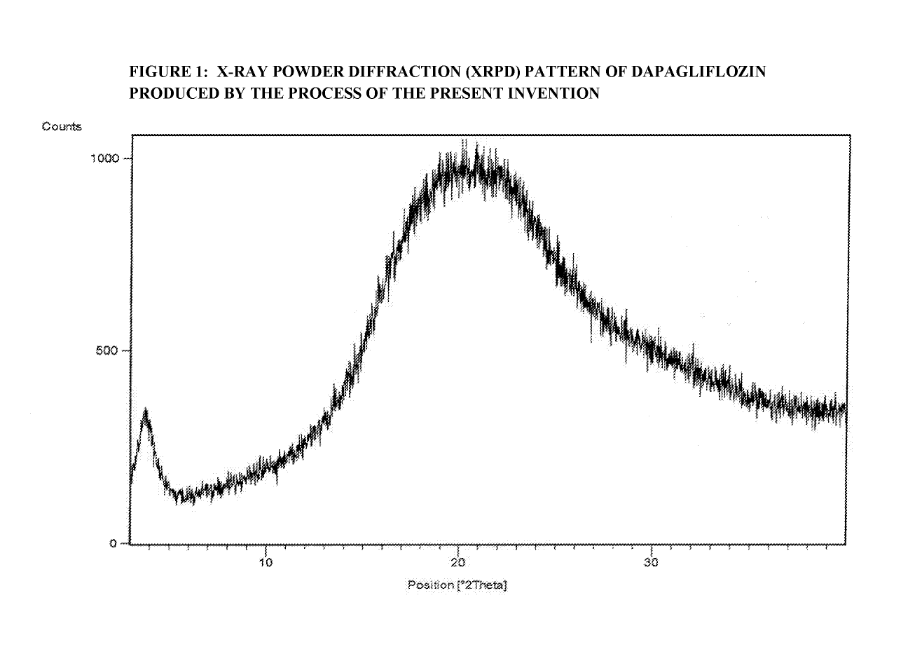

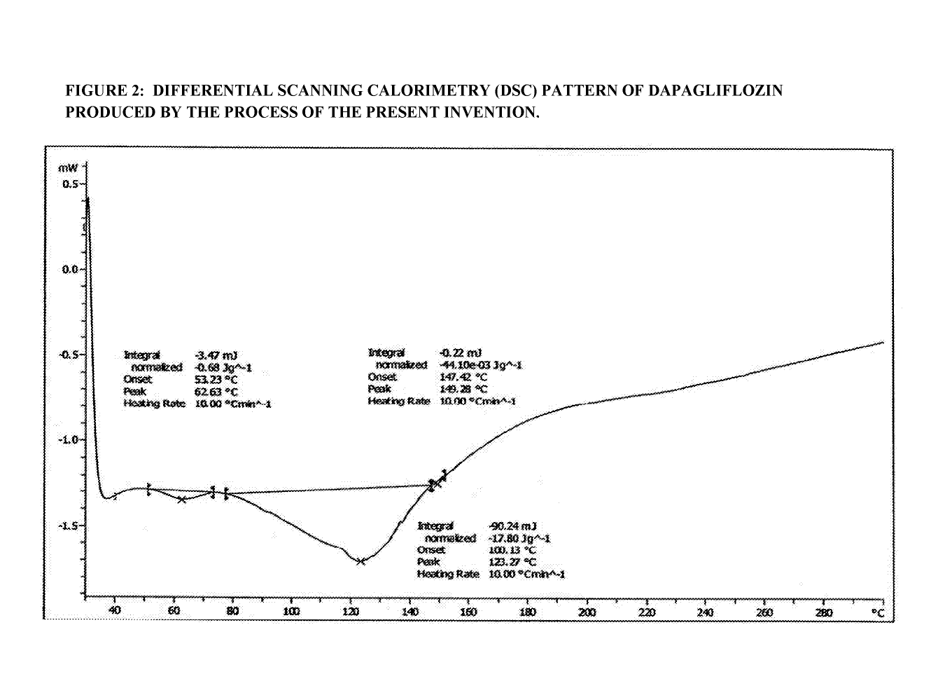

In another embodiment of the present invention, the dapagliflozin prepared by the process of the present invention is characterized by an XRPD pattern as depicted in FIG. 1 or a DSC as depicted in FIG. 2.

The compound of Formula III may be prepared by the process described in U.S. Pat. No. 6,515,117.

### Methods

XRPD of the samples were determined by using a PANalyitical® X'Pert Pro X-Ray Powder Diffractometer in the range 3-40 degree 2 theta and under a tube voltage and current of 45 Kv and 40 mA, respectively. Copper radiation of wavelength 1.54 angstroms and an X'celerator® detector were used.

The HPLC purity of dapagliflozin was determined using a Purospher® STAR RP-18e (150×4.6 mm), 3 μm column with a flow rate of 1.0 mL/minute to 1.5 mL/minute (flow gradient and organic gradient); column oven temperature: 25° C.; sample tray temperature: 25° C.; detector: UV at 225 nm; injection volume: 10 μL; run time: 60 minutes.

DSC was recorded using a Mettler Toledo® DSC 821e instrument.

The examples below are illustrated to aid the understanding of the invention but are not intended to and should not be construed to limit its scope in any way.

### REFERENCE EXAMPLE

### Preparation of dapagliflozin (Formula II)

A solution of lithium hydroxide monohydrate (1 g dissolved in 10 mL water) was added to a mixture of (1C)-2,3,4,6-tetra-O-acetyl-1,5-anhydro-1-[4-chloro-3-(4-ethoxybenzyl)phenyl]-D-glucitol (10 g), methanol (30 mL), and THF (20 mL) at 20° C. to 25° C. The reaction mixture was stirred for about 2 hours at 25° C. to 30° C. After completion of the reaction, the reaction mixture was concentrated under vacuum at 40° C. to 45° C. Ethyl acetate (100 mL) was added to the concentrated mixture and the reaction mixture was washed twice with brine solution (20 mL). The organic layer was separated and concentrated under vacuum at 40° C. to 45° C. to obtain a residue. The residue was dissolved in methyltertiarybutyl ether (30 mL) to obtain a solution. The solution was slowly added over hexanes (100 mL) at 5° C. to 7° C. The mixture was stirred for about 60 minutes at 5° C. to 7° C. and filtered under a nitrogen atmosphere to obtain a solid residue. The solid residue was washed with hexanes (10 mL) and dried under vacuum at about 40° C. to about 45° C. to obtain dapagliflozin.
HPLC Purity: 97.02%
Impurity at RRT 1.61: 0.84%
Other impurity: 1.49%

### EXAMPLE

### Preparation of Dapagliflozin (Formula II)

Methylamine (40% in water; 0.75 mL) was added to a solution of (1C)-2,3,4,6-tetra-O-acetyl-1,5-anhydro-1-[4-chloro-3-(4-ethoxybenzyl)phenyl]-D-glucitol (1 g) in methanol (20 mL) at 25° C. The reaction mixture was stirred for about 5 hours at 20° C. to 25° C. After completion of the reaction, the reaction mixture was concentrated under vacuum at 25° C. to 30° C. The pH of the reaction mixture was adjusted to 6-7 using hydrochloric acid (35% in water; ˜0.5 mL). Ethyl acetate (20 mL) was added to the reaction mixture and the mixture was stirred for about 10 minutes. The organic layer was separated, washed with water (10 mL), and dried using sodium sulphate (0.5 g). The organic layer was concentrated under vacuum at 40° C. to 45° C. to obtain a residue. The residue was dissolved in methyltertiarybutyl ether (MTBE; 5 mL) to obtain a solution. The solution was added to hexanes (10 mL) at 5° C. to 7° C. and stirred for 60 minutes to obtain a solid residue. The solid residue was filtered under nitrogen atmosphere and dried under vacuum at 25° C. to 30° C. to obtain dapagliflozin.
HPLC Purity: 99.92%
Impurity at RRT 1.61: 0.08
Other impurity: Not detected
XRPD as depicted in FIG. 1
DSC as depicted in FIG. 2

We claim:

1. A process for the preparation of dapagliflozin of Formula II, 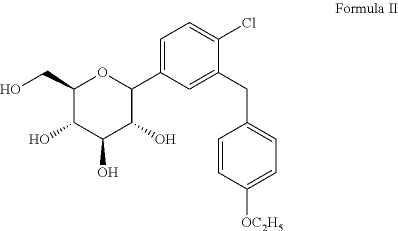 [SMILES: CCOc1ccc(Cc2cc(C3O[C@H](CO)[C@@H](O)[C@H](O)[C@H]3O)ccc2Cl)cc1]
wherein the process comprises the step of hydrolyzing the compound of Formula III 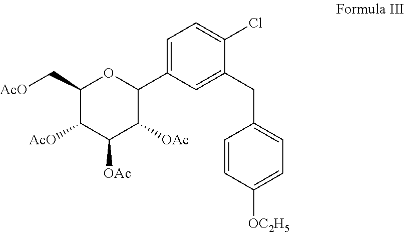 [SMILES: CCOc1ccc(Cc2cc(C3O[C@H](COC(C)=O)[C@@H](OC(C)=O)[C@H](OC(C)=O)[C@H]3OC(C)=O)ccc2Cl)cc1]
in the presence of an amine base.

2. The process according to claim 1, wherein the amine base is selected from the group consisting of ammonia, methylamine, dimethylamine, triethylamine, tert-butyldimethylamine, phenylethylamine, and diisopropylamine.

3. The process according to claim 1, wherein the hydrolysis is carried out in the presence of an alcohol solvent.

4. The process according to claim 3, wherein the alcohol solvent is selected from the group consisting of methanol, ethanol, n-propanol, isopropanol, butanol, and mixtures thereof.

5. The process according to claim 1, wherein the dapagliflozin produced is substantially free of an impurity detected at a RRT of 1.61, when measured by HPLC.

6. Dapagliflozin substantially free of an impurity detected at a RRT of 1.61, when measured by HPLC.

7. The dapagliflozin according to claim 6 characterized by an XRPD pattern substantially as depicted in FIG. 1 or a DSC substantially as depicted in FIG. 2.
